(19) United States
(12) Patent Application Publication
(10) Pub. No.: US 2021/0090721 A1
(43) Pub. Date: Mar. 25, 2021

(54) Systems and Processes to Guide Service Consumers through Everyday Services with Standardized Steps, Pairing Them With Service Providers to Best Fulfill Their Needs, Providing Expert Best Practice Advice To Ensure Their Needs Are Met, and by Predicting, Sensing, and Tracking Their Needs

(71) Applicants: Taylor A. Funk, Minneapolis, MN (US); Jackeline F. Krech, Minneapolis, MN (US); John Edward Krech, Minneapolis, MN (US)

(72) Inventors: Taylor A. Funk, Minneapolis, MN (US); Jackeline F. Krech, Minneapolis, MN (US); John Edward Krech, Minneapolis, MN (US)

(21) Appl. No.: 16/576,592

(22) Filed: Sep. 19, 2019

(51) Int. Cl. G16H 40/20; G06Q 30/08; G06Q 30/06; G06Q 10/06; G06Q 10/10; G06Q 30/04; G06Q 20/14; G06Q 40/08; G06Q 30/02; G16H 50/20; G16H 10/60; G06N 20/00; G01S 19/51; H04W 4/02

(52) U.S. Cl. CPC G16H 40/20; H04W 4/02; G06Q 30/0605; G06Q 10/06314; G06Q 10/06316; G06Q 10/10; G06Q 30/04; G06Q 20/14; G06Q 40/08; G06Q 30/0282; G16H 50/20; G16H 10/60; G06N 20/00; G01S 19/51; G06Q 30/08

(57) ABSTRACT

A service delivery system and method the applies universal steps for a service delivery, pairing a service consumer with a service provider based on their mutual profiles, providing service consumers knowledge of best practices, and sensing and tracking of additional service needs.

### BACKGROUND

People have more information at their disposal than ever before with the prevalence of smartphones. They are also more connected to these devices than any other piece of technology in history. However, there isn't an unified invention that both connects service consumers to everyday services and also guides them through the process of these services. Service consumers must look to various platforms including web sites, phone numbers, and physical locations to utilize services such as doctors visits, haircuts, buying a house, and countless other services. This inefficient process doesn't align with the speed and broad application of artificial intelligence capabilities that can be delivered through instruments such as smartphones to simultaneously schedule and pay for services.

Service consumers looking towards multiple locations for similar processes causes a lag in time that affects the service consumer and the service provider. Currently, if a service consumer wants to schedule a doctor's visit, they must call the office or schedule it using a web browser. There's a degree of likelihood the phone line will be busy. Mobile browsers can have many errors with loading web pages along with typographical errors while typing in small spaces that are meant to be on a laptop/desktop. These issues make it difficult for the service consumer to effectively and efficiently use these outdated processes. For example, the service consumer who wishes to buy a home may have first booked an appointment with a home inspector and then learns that their mortgage broker is not available for a week. In this scenario, the mortgage broker may have been available at the same time as the home inspector and the home inspector was available one day later. This would then require the service consumer to reschedule their appointment with the home inspector. This is an example of the added work for the service consumer.

These disconnected processes also help ensure that inconsistent processes are used and it is very difficult for the service consumer to put the big picture together. For example, a buyer of real estate will work with multiple processes and service providers that exist in silos, such as mortgage brokers, home inspectors, title companies, real estate agents and so on. Having these multiple silos often means submitting paperwork multiple times and requires multiple attempts to schedule and pay for services.

To make matters worse, it is very difficult to quickly determine if a service provider will match their needs. Service delivery can be broken down in to four key strategic drivers: will the service provider communicate in the manner that they expect, will the service provider show the compassion that they expect, will service provider deliver the reliable service they expect, and will the service provider deliver the value they expect. Frequently service consumer reviews and comments are shown en masse and it is very difficult for the service consumer to determine if the service provider matches their needs and expectations. Most service consumers have different needs and priorities, which makes their profile vary, maybe they prefer clear communication and explanations or maybe they prefer to just have quick and reliable service. Service providers typically have an ability to flex to more than one profile but are they able to flex to the service consumer's particular profile is the question. Another frequent problem is varying rating methods and scales that are often at summary level. This makes it difficult to compare service providers and to distinguish between them.

Service providers frequently deliver different levels of service and service consumers need to be equipped with the right information to ask the right questions. Some times the delivery of unsatisfactory service is because the the service consumer did not demand the best from the service provider. This could be due to the service provider being lazy or it could be because they are unaware. For example, if a person looking to buy a home that is located in a flood plain, the service consumer needs to be equipped to ask their insurance agent about the cost of this information but also be equipped to ask about the appropriate levels of coverage that are considered best practice. Another example, is the service consumer aware they should have their contractors sign lien waivers after completing a remodeling project. This is something many service consumers do not find out until they go to sell their house and it is difficult to find the contractor and get them to respond.

Completion of one service frequently leads to the need for another service. A particular service may consist of a multitude of sub-services. It is very difficult and complex for service consumers to see where they are going. Instead of following a guided path they feel like they are wandering through a maze unsure of which direction to go. For example, a service consumer buying a home may include sub-services with mortgage brokers, home inspectors, insurance agents, real estate agents and so on but the services required for the service consumer differ by the profile. For example, is this a first time home buyer, is it a cash buyer and so on.

In summary, these four themes of poor service delivery are what frequently prevents service consumers from having their needs met: non-standard and disconnected steps for service delivery, difficulty in selecting, scheduling appointments, and paying for service providers that can best match a service consumer needs, difficulty in ensuring best practices are followed and delivered, and difficulty in knowing the next steps and what direction to head next. It is the combination of these pillars that is needed.

### BRIEF DESCRIPTION

Having thus described the disclosure in general terms, reference will now be made to the accompanying drawings, which are not necessarily drawn to scale, and wherein:

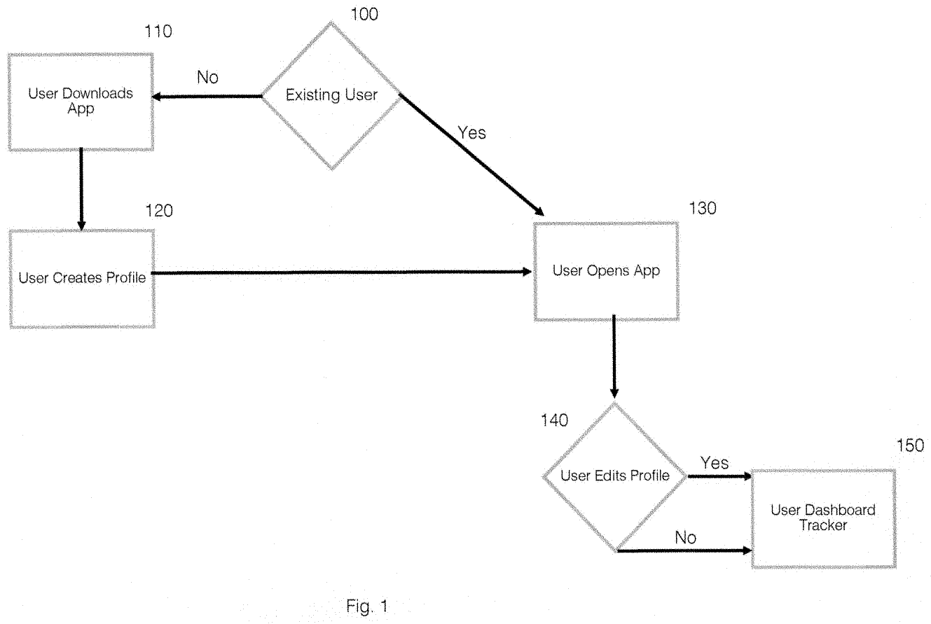

FIG. 1 provides an illustration of an exemplary embodiment according to the first stage of the present disclosure;

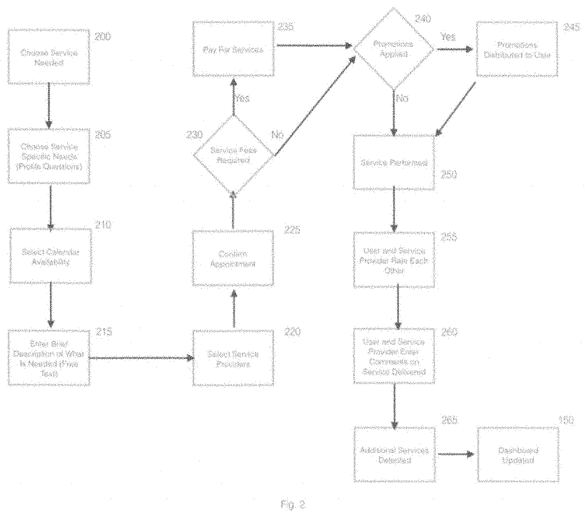

FIG. 2 provides an illustration of an exemplary embodiment according to the second stage of the present disclosure;

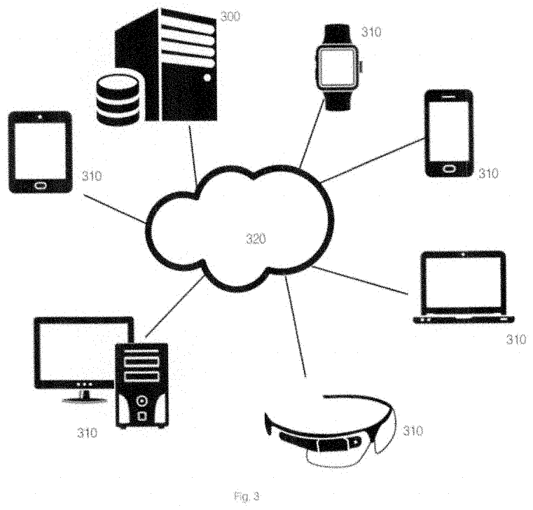

FIG. 3 provides an illustration of an exemplary embodiment of the present disclosure;

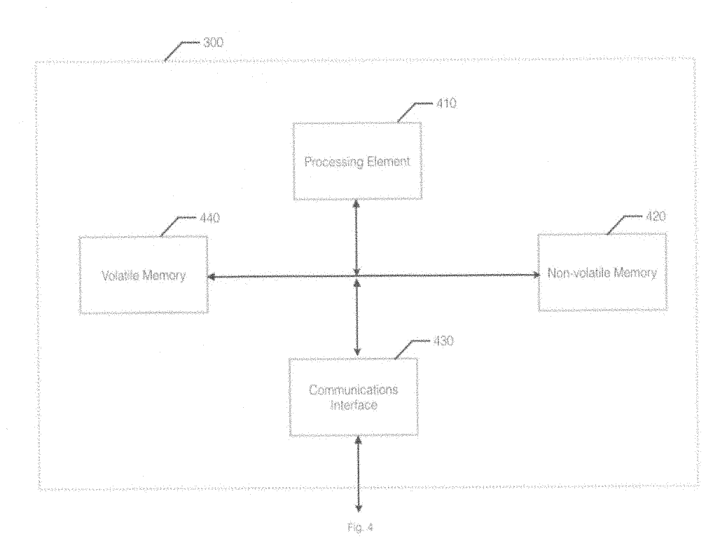

FIG. 4 provides an illustrative schematic of an exemplary management computing entity according to one embodiment of the present disclosure;

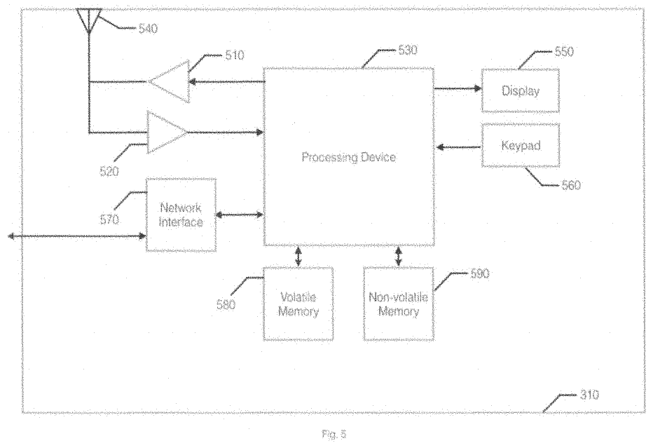

FIG. 5 provides an illustrative schematic representative of an exemplary mobile computing entity that can be used in conjunction with embodiments of the present disclosure;

### DETAILED DESCRIPTION OF THE INVENTION

Having these four pillars of service delivery are what overcomes the four themes outlined above and ensures the service consumers needs are met: Standard universal steps for a service delivery; Pairing, scheduling, and paying for appointments with service providers that best match their priorities for their profile; Providing knowledge and expertise to ensure best practice services for their profile; and Sensing and tracking of additional services. It is the combination of these pillars that is needed.

In one embodiment the invention uses a mobile phone app that functions as a hub for the broad array of services people use daily through a series of standard universal steps. Through the app, service consumers will be able to schedule appointments for services in an organized, detailed, and pain-free process. The app will have a function that allows service consumers to give an input and then the algorithm points them in the right direction on where to proceed next. For example, if a person (a service consumer) is feeling ill and wants to see an appropriate doctor (a service provider), they can type or speak in “headache” in the app and a prompt to schedule an appointment with a doctor will be available. This could lead to appointments being made within the hour as doctors who have capacity can set their status to see patients as “open” so they can receive a patient right away. This hub connects the various services and service providers into a centralized location.

This cuts down on waiting room time in the specific doctor example. However, this same concept can be applied to many different service providers, such as seeing a home with a registered real estate agent (a service provider). The buyer (a service consumer) would indicate they are looking to see a home and a nearby agent who has their status as “available” would be able to show the home to the buyer. It's a more instantaneous way of receiving/giving a service. Depending on the settings by the seller (a service consumer), an appointment could be instantly booked however if a seller has certain conditions, the nearby agent would negotiate a showing appointment that meets both service consumer's needs.

In another embodiment, the invention pairs the service consumer with a service provider for their profile and ensure best practice service delivery. Service delivery can be broken down in to four key strategic drivers: will the service provider communicate in the manner that they expect, will the service provider show the compassion that they expect, will service provider deliver the reliable service they expect, and will the service provider deliver the value they expect. These strategic drivers are built into the app as well as the next level down to the tactical drivers. For example, a potential tactical driver for the strategic driver regarding providing reliable service could be make things simple. Another example tactical driver for the strategic driver communicate in the manner I expect could be provide clear information how to get most of the service delivered. Each of the tactical drivers is a question with the creation of a service consumer profile and together they roll up to determine their likelihood to recommend the services of a service provider. This profile as well as their ratings of any prior services experiences is what is used to pair service consumers with service providers are best predicted to have a likelihood to satisfy the service consumer and make them what is known as a Net Promoter Score (NPS) Promoter. This also helps the service providers by pairing with service consumers who they are most likely to satisfy with their strengths and capabilities.

The Net Promoter Score (NPS) is an index ranging from −100 to 100 that measures loyalty of a first party (e.g., service consumer) to a second party (e.g., service provider). The NPS can provide businesses with a snapshot of loyalty and engagement within or toward their company. The NPS is based on a single question: “on a scale of 0-10, how likely is it that you would recommend [company name] to your friends, family or business associates.” This question is a “Likelihood to Recommend” of LTR. Respondents who indicate a 6 or below are “detractors,” those who indicate a score of 7-8 are “passives,” and those who indicate a score of 9-10 are “promoters.” Calculating the NPS from the survey responses involves a procedure of subtracting the percentage of detractors from the percentage of promoters.

In another embodiment, the invention first pairs the service consumer with service providers have achieved a premium status meaning that they have consistently received superior feedback over an extended period of time for the service consumer's key strategic and tactical drivers. For example, a LTR of 9 or above for 12 months with zero cancellations.

In another embodiment, the invention pairs the service consumer with a service provider based a transaction fee such as booking fee, a showing fee, or a co-pay. Within this embodiment, the service provider may be permitted to utilize a minimum and a maximum price for situations where an exact price cannot be provided. In another embodiment, the fees are collected from the service consumer at the time of booking and distributed to the service provider at the time of service delivery. In another embodiment, the invention can also pair the service consumer with a service provider based on a mutual sharing of a commission. For example, a buyer of real estate may receive 15% of the commission that a real estate agent received.

In another embodiment, the invention pairs the service consumer with the service provider for their profile based on the relative proximity to each other. For example, a service consumer could search for service providers on a map in the app by choosing a radius, such as within one mile, to see all the available service providers and their relative ratings within that area. In another example, a service consumer could draw a polygon shape on a map to see all available service providers within that area. In another embodiment, the service providers location could be based on current mobile phone location.

In another embodiment, the invention uses artificial intelligence to provide knowledge and expertise to ensure best practice services for service consumer profile. Service providers frequently deliver different levels of service and service consumers need to be equipped with the right information to ask the right questions. Sometimes the delivery of unsatisfactory service is because the service consumer did not demand the best from the service provider. This could be due to the service provider being lazy, inexperienced, or it could be because they are unaware. For example, if a service consumer looking to buy a home that is located in a flood plain, the service consumer needs to be equipped with the knowledge to ask their insurance agent about the cost of this information but also be equipped to ask about the appropriate levels of coverage that are considered best practice. Another example, is the service consumer may not be aware they should have their contractors sign lien waivers after completing a remodeling project. This is something many service consumers do not find out until they go to sell their house and it is difficult to find the contractor and get them to respond. Having this knowledge quickly and easily available helps ensure best practices are followed.

The efficiencies of having two-way form of communication between the service provider and the service consumer to instantaneously match them is great. What if the service consumer has no clue as to how the process of receiving a service works (such as buying a home)? The app will also have a feature that walks the service consumer through the steps necessary to complete the service and any sub-services.

In another embodiment the invention senses and tracks the need for additional services. Completion of one service frequently leads to the need for another service. A particular service may consist of a multitude of sub-services. It is very difficult and complex for service consumers to see where they are going. Instead of following a guided path they feel like they are wandering through a maze unsure of which direction to go. This is why it important to sense additional services and guide service consumers along the various options step by step.

In another embodiment, the invention tracks and provides reminders for regular or recurring appointments. For example, reminding the service consumer two months before they are due for a regular dental check-up.

In another embodiment, the invention tracks the completed documents of each service. For example, taxes, lien waivers, bank statements, insurance documents, medical records and/or the like. In another embodiment these documents can be uploaded from prior records. By storing these records in the appropriate locations, records can automatically be shared with service providers. This makes the process more efficient.

In another embodiment the invention tracks the purchase of services across multiple service provider web sites and/or calendar software modules. The embodiment comprises a calendar cart software module which records services and dates selected by the service consumer from the multiple web sites and/or calendar software modules in a personal calendar cart. In a further embodiment, the calendar cart remains persistent over multiple service consumer sessions, wherein the services are selected by the service consumer. The embodiment also comprises an ordering software module that enables the service consumer to purchase the services for the dates and times recorded within the calendar cart, including items selected from web sites and/or calendar software modules of multiple different service providers, by placing a single order.

In another embodiment, at least some of the services are from service providers that require an appointment with a confirmed date, time, and duration before processing payment.

In another embodiment, simultaneous calendar booking prevents having more than one appointment scheduled at the same time and also can find the optimum time for all appointments to be scheduled without the need to reschedule and adjust schedules. Simultaneous calendar booking can also maintain a sequential order of meetings. For example, a buyer of real estate (a service consumer) should first meet with a mortgage broker (a service provider) to get pre-approved and then meet with a real estate agent (a service provider) to look at homes in their price range.

As an example, a service consumer looking to buy a property would start with the pre-approval stage (with a lender), then meet with an agent (using the app in the aforementioned process), sign a purchase agreement then go through the inspection phase (hire an inspector). The app leads the buyer through each stage in the process and puts them in contact with the correct professionals at the right time. The app also senses and predicts which services the buyer may or may not need. The services required for the service consumer differ by the profile. For example, is this a first time home buyer, is it a cash buyer and so on. Predicting and sensing the services required and bringing them to the service consumer in a step by step manner ensures that the process is simple and the services delivered meet the needs of the service consumer.

### PROBLEM INVENTION SEEKS TO SOLVE

As mentioned in the previous section, one embodiment of the invention uses a mobile phone app that functions as a hub for the broad array of services people use daily through a series of standard universal steps. Currently, service consumers must look to many locations, both physical and online, in order to set their appointments. This process creates chances for inefficiencies such as the service providers' phone line being busy or arriving at a physical location only to wait for an additional period of time because the service provider is busy with another customer. The waiting period doesn't match with the technological efficiencies that have been created within the smartphone space.

To add on to these inefficiencies, often times service consumers don't know what stage in the process they're in for rendering the services. In another embodiment mentioned in the previous section, the invention senses and tracks the need for of additional services. Having a tracker that updates service consumers in which steps have been completed, and which remain, makes the process less of a hassle. New service consumers looking for a service for the first time (first-time home buyers) will find it useful to have the steps laid out in front of them and which step they're on. Often, they're unsure on the timing for which professionals they need to be in contact with. This can add to the stress level that goes along with a service need. Service consumers with experience in the service field they're needing will still find it useful to know the timing for when they can receive a service, whether it be the same day or two weeks from now, based off the timeline of stages the app provides. Both types of service consumers will know they are receiving time from the best services provider as the rating system can inform them of the quality of the service provider.

Within the embodiment to provide a series of universal steps include but are not limited to:
1. Choose what service is needed
2. Identifying the specific need within the service
3. Check calendar availability (can be within the hour or weeks out)
4. Brief description of what's needed (so the service provider is aware)
5. Schedule/confirm appointment
6. Pay for services (linked to account/insurance providers info.)
7. Service consumers/providers both rate one another
8. Sense and track the next steps to go forward in the process

The relationship between these steps as well the embodiments previously outlined are discussed below.

The first step, choosing the service needed, is straightforward. The service consumer will open the app and a dashboard will appear with a search bar embedded. If looking for a new service, the service consumer will type or speak in a quick description so the app can provide results for that service. The various service providers will all be linked to the app through the database, allowing their information to be displayed following this service consumer input. Within this dashboard, service consumers can also monitor the progress of the other services, if they've already established them. As an example, a service consumer whose child wakes up ill one morning would type in the search bar something along the lines of “physician” or “urgent care” and nearby clinics with availabilities would appear. If this same service consumer is in the process of being pre-approved by a mortgage banker to buy a house, this status would be displayed in the dashboard tracker. The service consumer can easily shift between creating new service requests and monitoring the progress of their current ones. Seeing the additional services is within the embodiment to predict, sense, and track additional services.

Within the first step, is the uploading documents to make reaching out service providers more efficient. Examples of these documents include but are not limited to: Financial Statements, Health Insurance Cards, Medical records, Tax record, Employment records, Social media, Email, Text, Calendar, Business calendar, Ride sharing such as Uber, Lyft, and/or the like.

Choosing the specific need within the service is the second step in the overall process. Here, service consumers can differentiate between broader categories that are found in various industries. It is here that the embodiment mentioned in the previous section, the invention pairs the service consumer with service provider for their profile and best practices. Going back to the ill child example, a service consumer would select a child physician because the child is sick or request a certain type of doctor (e.g. general care) to see the child. The service consumer can also filter on the ratings to choose the minimum rating they desire (keeping in mind the more highly rated, the more in demand the service provider will be). The purpose of this stage is to filter out the many search results that are connected to a particular service. It narrows the information down to better suit the needs of the service consumer. The service provider will give their price, in some cases a minimum and a maximum, for service in this stage.

Within this previously mentioned embodiment, the invention pairs the service consumer with a service provider for their profile and ensure best practice service delivery. Service delivery can be broken down in to four key strategic drivers: will the service provider communicate in the manner that they expect, will the service provider show the compassion that they expect, will service provider deliver the reliable service they expect, and will the service provider deliver the value they expect. These strategic drivers are built into the app as well as the next level down to the tactical drivers. For example, a potential tactical driver for the strategic driver regarding providing reliable service could be make things simple. Another example tactical driver for the strategic driver communicate in the manner I expect could be provide clear information how to get most of the service delivered. Each of the tactical drivers is a question with the creation of a service consumer profile and together they roll up to determine their likelihood to recommend the services of a service provider. This profile as well as their ratings of any prior services experiences is what is used to pair service consumers with service providers who are best predicted to satisfy the service consumer and make them have a likelihood to make them known as a Net Promoter Score (NPS) Promoter. This also helps the service providers by pairing with service consumers who they are most likely to satisfy with their strengths and capabilities.

The Net Promoter Score (NPS) is an index ranging from −100 to 100 that measures loyalty of a first party (e.g., service consumer) to a second party (e.g., service provider). The NPS can provide businesses with a snapshot of loyalty and engagement within or toward their company. The NPS is based on a single question: “on a scale of 0-10, how likely is it that you would recommend [company name] to your friends, family or business associates.” This question is known as “Likelihood to Recommend” or LTR. Respondents who indicate a 6 or below are “detractors,” those who indicate a score of 7-8 are “passives,” and those who indicate a score of 9-10 are “promoters.” Calculating the NPS from the survey responses involves a procedure of subtracting the percentage of detractors from the percentage of promoters.

In another previously mentioned embodiment, the invention first pairs the service consumer with service providers have achieved a premium status meaning that they have consistently received superior feedback over an extended period of time for the service consumer's key strategic and tactical drivers. For example, a LTR of of 9 or above for 12 months with zero cancellations.

In another previously mentioned embodiment, the invention pairs the service consumer with a service provider based a transaction fee such as booking fee, a showing fee, or a co-pay. In another embodiment, the fees are collected from the service consumer at the time of booking and distributed to the service provider at the time of service delivery. Within this embodiment, the service provider may be permitted to utilize a minimum and a maximum price for situations where an exact price cannot be provided. In another embodiment, the invention can also pair the service consumer with a service provider based a mutual sharing of a commission. For example, a buyer of real estate may receive 15% of the commission that a real estate agent received.

In another previously mentioned embodiment, the invention pairs the service consumer service provider for their profile based on the relative proximity to each other. For example, a service consumer could search for service providers on map in the app by choosing a radius such as within one mile to see all the available service providers and their relative ratings within that area. In another example, a service consumer could draw a polygon shape on map to see all available service providers within that area. In another embodiment, the service provider's location could be based on the current model phone location.

The third step is matching the availability of the service consumer with that of various options for service providers. Both the service consumer and the service provider will have a calendar feature where that will block off the time for the appointments they have scheduled. This way the service consumer can see what availabilities the service provider has to best align with their own schedule. In another previously mentioned embodiment, service consumers will be able to simultaneously see appointments across multiple service providers. To make sure the service consumer is legitimately requesting a service, information such as that on an information card will be asked for. For a service in which the service provider isn't paid right away, there will be a fee charged if the service consumer doesn't show up to the appointment (as we already have their credit card/bank account information). The service consumer will not be able to schedule another appointment until this fee has been paid. The timeframe on services can be immediate (so long as the service provider has an opening) through months in advance.

The fourth step is a brief description of what the specific service request is. The service consumer will give a one to two sentence overview of what their need is. The overview gives the service provider ample time to prepare for the request, allowing them to already have part of the solution thought out before the service consumer even meets with them. This creates even more efficiencies on both ends, leading to more time being spent elsewhere. Currently, service consumers have to describe the need multiple times before anything is even being done to remedy it. This phase speeds up the overall process on both ends.

The fifth stage, scheduling/confirming the appointment, is exactly what it sounds like. After the searching, identifying the specific need, checking availabilities, and giving a brief overview, the service consumer is ready to confirm the appointment. In another previously mentioned embodiment, service consumers will be able to simultaneously confirm appointments across multiple service providers with a single confirmation. After it's confirmed, the appointment will appear on the service consumer's and the service provider's calendar.

Paying for services, the sixth stage, occurs after the service has been provided. The service consumer has either credit/debit card on file or links the app to their bank account. In another embodiment payment can be made through a digital wallet mobile platform such as Venmo, Apple Pay, and the like. In another previously mentioned embodiment, service consumers will be able to simultaneously pay for appointments across multiple service providers with a single payment.

Issues with the service provided are dealt within this stage to identify if the service provider did their job and whether or not they should be paid adequately. For services where third party payers are involved (health insurance) the service consumer will enter that information in who will in turn pay the service provider, based off their agreement. Within a previously mentioned embodiment this information could be electronically retrieved from the documentation the service consumer loaded into their profile. For example, health insurance and medical records. Within this stage, a previously mentioned embodiment of the invention is to provide knowledge and expertise to ensure best practice services for the service consumer's profile.

Service providers frequently deliver different levels of service and service consumers need to be equipped with the right information to ask the right questions. Some times the delivery of unsatisfactory service is because the service consumer did not demand the best from the service provider. This could be due to the service provider being lazy or it could be because they are unaware or they are less skilled. Having knowledgeable service consumers is an opportunity for the services providers to deliver their best at all times and perhaps learn more by having more demanding service consumers. For example, if a person looking to buy a home that is located in a flood plain, the service consumer needs to be equipped to ask their insurance agent about the cost of this information but also be equipped to ask about the appropriate levels of coverage that are considered best practice. Another example, is the service consumers aware they should have their contractors sign lien waivers after completing a remodeling project. This is something many service consumers do not find out until they go to sell their house and it is difficult to find the contractor and get them to respond. Having this knowledge creates more informed reviews in the next stage.

The seventh stage is where the service consumer rates the service provider and the service provider rates the service consumer. It will be a 10-star system, with 10 being the most outstanding for each of the tactical and strategic drivers. Service delivery can be broken down into four key strategic drivers: will the service provider communicate in the manner that they expect, will the service provider show the compassion that they expect, will the service provider deliver the reliable service they expect, and will the service provider deliver the value they expect. These strategic drivers are built into the app as well as the next level down to the tactical drivers. Each strategic driver contains tactical drivers. For example, a potential tactical driver for the strategic driver regarding “providing reliable service” could be to “make things simple”. Another example of tactical driver for the strategic driver “communicate in the manner I expect” could be to “provide clear information how to get most of the service delivered”. Each of the tactical drivers is a question after the completion of the service delivery with the 10 star system with 1 star being and very unsatisfied and 10 being very satisfied. Each service can consist of separate strategic and tactical drivers are most important in predicting whether a service consumer will have a likelihood to be a Net Promoter Score (NPS) Promoter. This helps the service providers by pairing with service consumers who they are most likely to satisfy with their strengths and capabilities. Most importantly, this helps service consumers be paired with service providers who can satisfy their needs.

The Net Promoter Score (NPS) is an index ranging from −100 to 100 that measures loyalty of a first party (e.g., service consumer) to a second party (e.g., service provider). The NPS can provide businesses with a snapshot of loyalty and engagement within or toward their company. The NPS is based on a single question: “on a scale of 0-10, how likely is it that you would recommend [company name] to your friends, family or business associates.” This question is known as the “Likelihood to Recommend” or LTR. Respondents who indicate a 6 or below are “detractors,” those who indicate a score of 7-8 are “passives,” and those who indicate a score of 9-10 are “promoters.” Calculating the NPS from the survey responses involves a procedure of subtracting the percentage of detractors from the percentage of promoters.

The strategic and tactical drivers have been statistically correlated to NPS using a number of regression modeling techniques known to one skilled in the art. These techniques include but are not limited to linear stepwise regression, partial least squares regression, and Bayesian regression. Within this previous mentioned embodiment to pair the service consumer with a service provider, it is important to stress that this is a two way review process of the service consumers and of the service providers not only match service consumers to service providers but vice versa to match service providers to service consumers. Within this embodiment, this matching including using previous service consumer profiles for the service provider that are most similar to the service consumer to provide additional insight.

The final stage, the eighth one, takes the outcome of the service and gives the service consumer a next steps feature via another embodiment the invention senses and tracks the need for additional services using artificial intelligence. It takes into account the summary that the service provider gives and uses predictive analytics to determine next steps. Say a person went to a doctor and it was determined the have diabetes (the doctor said this in the description). The app would then provide steps the service consumer should take such as medication needed, where to get it, dietary guidelines, exercise guidelines, etc. all while pointing them in the right direction on where they can find said service (essentially bringing them back to stage one, just for a new service). The value of this stage is that it organizes the service consumer's next steps so they don't have to research them on their own. It also provides them with reminders on the next steps they have to do (blood testing in a month) and asks if they want it scheduled right away. The service providers will receive these repeat service consumers if the ratings matched and its within their capabilities, leading to repeat customers.

Within the final stage, the previous mentioned embodiment of the invention tracks the purchase of services across multiple service provider web sites and/or calendar software modules including any new services that are recommended. The previously mentioned embodiment comprises a calendar cart software module which records services and dates selected by the service consumer from the multiple web sites and/or calendar software modules within a personal calendar cart. One benefit of this feature is that it enables the service consumer to perform a single “check out” to purchase services from multiple web sites and/or calendar software modules. Additionally, this feature allows the app to accurately track and credit each associate, on a per-product-sale basis, that has referred a customer. For example, if, upon “check-out” from the app, the customer has three booked appointments in the calendar cart, each of which resulted from a referral from a different service provider website and/or calendar software modules, each service provider will be credited for its respective referral.

The steps described above are accompanied with diagrams that visually display each of their processes:

FIG. 1 maps out what service consumers will see when they first open the app along with an overview of the main steps. New service consumers will be prompted in 120 to create a profile by entering their email address and a password, state issued identification, banking information, address, phone. They will also be asked a series core strategic and tactical driver questions to determine their initial service provider profile. Depending the services to be delivered, additional steps could be included, such background check etc . . . Existing service consumers will sign in at 130, displaying the dashboard listing their current progress in 150 for any services still pending along with a search function that will allow them to request new services.

### HOW THE INVENTION FUNCTIONS

With the object of the invention being to efficiently provide services to service consumers and to guide them through the process, the invention must have great software behind it, which it does. The software in place makes sure the availabilities are accurate and updated in a seamless fashion. The software is not only functional, but it also lays out each stage in scheduling and receiving a service in a way that makes it easy for service consumers to follow, pairs them with service providers that have the highest likelihood to meet their needs, provides expert best practices, and predicts the future needs. The combination of these four elements is what sets the app apart and what will truly drive value for the service consumer by coordinating, scheduling, and paying for multiple service providers simultaneously.

The service provider will also utilize the app due to the simple fact that service consumers will flock to the app, for aforementioned reasons, and that it improves their time efficiencies as well. Having information before even meeting with a service consumer gives them more time to prepare for that particular client, whilst meeting with more of them due to consolidation of information being shared in one location. The app has functional, accurate, and organized software that will make the lives of the service consumers and service providers more organized and decrease the time for receiving or giving services.

The first stage operates as an initial dashboard in FIG. 1 item 150 to highlight which stage of current services the service consumer is in (whether it be the appointment is scheduled for a doctor's visit or waiting on passport verification if traveling) along with a search feature enabling the service consumer to start the simple process of requesting a new service to be added. The key elements of this stage are to provide detailed progress reports of current services and to make the search function easy and accurate for the service consumer. The software embedded in the app allows for both these crucial elements to seamlessly work with one another to provide whichever detail that the service consumer is looking for.

Selecting a specific service is the second stage in FIG. 2 item 200. This stage is used to refine and filter what the service consumer is looking for. After the service consumer inputs a key word in the search function, the app will generate results based off this input. The service consumer can filter on categories like location, rating quality, and availability in 205. This stage is necessary to provide the service consumer with efficiencies for receiving the service. By filtering on service providers with immediate openings, the service consumer can quickly fill their need. By filtering on rating, the service consumer can be assured they are receiving the top service. The app will utilize the service consumer and the provider's locations to aid in filtering on location should the service consumer want to filter on it. This involves the use of GPS. Rating quality is based off an algorithm found within the app that takes prior service consumer's ratings into account (this process is elaborated more in stage seven, where the service consumers and providers rate one another).

At the time of enrollment as a prospective service provider, service providers will create a profile including their trust accounts and financial institution accordingly. They will also fill out any insurance provider identification as well as set price and cancellation and change policies. They will also provide links to calendar system. Finally, they will answer service profile questions on how they view themselves. Service consumers will be able to see how service providers rate themselves and how their previous customers through app rate them.

The third stage, checking the calendar is a crucial step in connecting the service consumer with the service provider in 210 It is here where the app uses its own calendar feature that takes input from the service consumer on what specific time/date they're looking to use a service. This time preference from the service consumer filters to the calendar on the service provider's end to match up time periods/dates. The availabilities will appear to the service consumer where they can then confirm the time that matches with their preferences and the service provider's openings in schedule.

If the service provider doesn't have an availability within the time period that the service consumer desires, they will be prompted with options to either select a new time period or to seek a different service provider. For selecting a new time period, the app will tell the service consumer when the service provider has their next availability. In the case of seeking a new service provider, the app will provide suggestions on different service providers based on the criteria that the service consumer input in step number two (such as rating or location).

Within stage three, the app will also have a feature where the service provider can block off time when they're not open or don't want to receive any customers from the service consumer base. This is especially necessary so a service provider's whole calendar isn't booked full with service consumers trying to book their service requests. There will be an option to select windows of time that is completely customizable to the service provider. As an example, (the possibilities are completely up to the service provider) one service provider might want to have their window be open for 12 hours Monday-Thursday. Another might choose to work for 4 hours a day, only on the weekend. Those that are open for more hours will often time get more service consumers just based on sheer numbers/opportunities to match with a service consumer's time preference. However, some who are the highest rated might find themselves in most demand so they could adjust their hours/pricing accordingly.

If scheduling with multiple service providers, the service consumer will see the optimum time periods to simultaneously schedule the appointments. If there is a specific sequence for the appointments, the service consumer what order the appointments must be.

Stage number four in 215, providing a description of the issue, is a simple way to create efficiencies on both the service consumer's end and the service provider's end. A quick two sentence summary describing the problem and what the service consumer seeks goes a long way in aiding the service provider to help out in the best way possible. This way the service provider can start to plan out a course of action to help the service consumer long before the service consumer even meets the service provider face to face. This creates less time waiting for the service consumer and can create more inflow of service consumers for the service provider.

As an example, the founder of a start-up company wants to meet with a lawyer about incorporating their business. They have no clue what the process is and how much money it could be potentially be. They have already selected one of the top rated lawyers and connected on a time based off the first three stages (mentioned above). Meeting with the lawyer is going to be extremely beneficial even just meeting as is. However, if the service consumer provides the lawyer with a few details such as what type of start up it is, where they want to incorporate, whether they want to be a LLC or a C-corp, etc. this can tremendously improve the productivity and the meeting and cut the meeting time down. The meeting time being shortened saves value for the service consumer as most of these lawyers are paid hourly (along with many more professions). The service provider also has the potential to capture more revenue as they can work with more clients in a given amount of time and gain more money through numbers as opposed to individual sessions.

Stage five is officially scheduling and confirming appointment in 225, is simply what it sounds like. Here, the service consumer has already selected the service, selected an available time, and described the service needed in a little more depth. The first initial tracking screen has already told them which stage in the process the service consumer is in. After the service consumer confirms the time (that is also available on the service provider's calendar) then this meeting will be set for whatever time period was selected. This could be the same day or months in advance. The meeting will then appear on the service provider's calendar. If scheduling multiple appointments, the service consumer selects and confirms multiple appointments simultaneously with a a single calendar “check-out”.

To caveat the notion that service provider's will be caught off guard with last minute requests, the app fulfills these gaps. If a service provider has made themselves available for a block of time, but no one has booked yet then the service provider would be waiting for that spot to be filled right anyway. They'll be happy that someone has scheduled to meet with them during a time period when no one had yet done so.

The app will also provide updates to both the service provider and the service consumer. These updates can come in the form of cancellations, changes, interim steps that may be necessary, and reminders of an upcoming meeting. These reminders can include the enforcement of cancellation and change fees. For example, service providers cannot cancel appointments without losing premium status for a period of time, for example 12 months. Service providers set period of refund and penalty fee within their profile and this is communicated to the service consumer prior to scheduling the appointments. A provider will set how many days/hours they allow cancellations and/or changes without this affecting the service consumer. The same applies to the service consumer. These safeguards are just to make sure no one is deploying fraud or scams to block off competitor's schedules and then simply cancelling appointments. There are some small ramifications if a cancellation or changes does occur within 24 hours of a meeting (regardless of the settings). Interim steps are items an service consumer can perform prior to meeting with the service provider without their aid.

Stage six in 235, paying for the service, is obviously one of the most important of the eight. It's the reason why service providers do what they do and why customers must seek out service providers in the first place. However, this stage can be a little nuanced based off which industry the transaction took place in (i.e. real estate as opposed to medical). Some industries the norm is that the service provider gets paid following the completion of some event (in the case of real estate the buying or selling of a home). However, in some industries the service provider is paid even before meeting with the client. The app will understand these different trends in each industry and make sure the norm is followed. In cases where insurance becomes a factor, the app will collect the service consumer's insurance information prior to the meeting so payment can be collected (which would be an interim step in stage five).

The app tracks the purchase of services across multiple service provider websites and/or calendar software modules. The embodiment comprises a calendar cart software module which records services and dates selected by the service consumer from the multiple web sites and/or calendar software modules in a personal calendar cart. As discussed above, the present invention provides a system for maintaining a unified calendar cart that stores service information associated with service referrals from multiple service provider web sites and/or calendar software modules, and keeps track of the sources of such referrals. One benefit of this feature is that it enables the service consumer to perform a single calendar “check-out” to schedule and purchase services from multiple service provider web sites and/or calendar software modules. Additionally, this feature allows the app to accurately track and credit each service provider, on a per-appointment basis. For example, if, upon “check-out” from the app, the service consumer has three booked appointments in the calendar cart, each of which resulted from a referral from a different service provider website and/or calendar software modules, each service provider will be credited for its respective transaction fee.

Service providers will be able to customize payment somewhat. Say a real estate agent feels they're extremely well-versed in the current marketplace and they want to charge money just for showing buyers homes (in addition to the commission at the end). They can choose a price simply for taking service consumers on showings. This might seem like a flawed feature, but these service providers will know that not as many service consumers will be willing to pay that way and so they're list of clientele will go down. If their list goes down, they must achieve a spectacular rating so that people will be willing to pay them per showing knowing that they do a good job of connecting buyers with the homes they want to buy. In addition, service consumers can simply filter out these agents who pay per showing to cater it to more of the traditional style.

The seventh stage, the service consumer and the service provider rate one another, in 255 occurs after the service has been performed. The app will use a 10 star system, with 10 being the most outstanding based on the services strategic and tactical drivers. Each of the tactical drivers is a question after the completion of the service delivery with the 10 star system with 1 star being and very unsatisfied and 10 being very satisfied. In addition to these questions is the core Net Promoter Score (NPS) is an index ranging from −100 to 100 that measures loyalty of a first party (e.g., service consumer) to a second party (e.g., service provider). The NPS is based on a single question: “on a scale of 0-10, how likely is it that you would recommend [company name] to your friends, family or business associates.” This question is known as “Likelihood to Recommend” or LTR. Respondents who indicate a 6 or below are “detractors,” those who indicate a score of 7-8 are “passives,” and those who indicate a score of 9-10 are “promoters.” Calculating the NPS from the survey responses involves a procedure of subtracting the percentage of detractors from the percentage of promoters. These scores are then used as a way to filter on the quality of service that the service consumer and service provider are looking for, respectively. There will be a feature that shows how many reviews either one has received throughout their time using the app. This will allow others to determine if a score was simply a one-off or if that's how the average has been for multiple reviews. This allows for further filtering for both the service consumer and the service provider.

This review process also has a private and a public component. A public review will be displayed for everyone to see. A private one is sent only to the service consumer or service provider. Often times, these will be ways to improve or tips that they can use. Distinguishing these can allow for a more fair assessment that doesn't overly deflate or inflate a particular review.

The eighth and final stage is determining the next steps for the service consumer in 265 (and service provider, if necessary). When the meeting is concluded, the app prompts the service provider to briefly summarize the meeting so the app can determine the next steps necessary. This is an extremely useful stage in the process and gives the app a lot of functionality throughout industries. Being able to to provide the service consumer with prompts and suggestions for the next stage is invaluable in saving them time and being a one-stop app for services. Any required documentation for the next steps can be electronically submitted to the services providers with the service consumers authorization and the service consumer will be prompted for any missing documentation using a document retention software module.

As an example, in the medical industry, if a doctor diagnoses the service consumer with diabetes, then the doctor will put this in the summary at the end of the meeting (along with specifics like the type, severity, etc.). The app will then prompt the service consumer with notifications about dietary restrictions, where to go to find the proper medicine, and other items such as exercise programs and nutritional health books. If the service consumer needs to see continued treatment from a doctor/specialist the app will prompt the service consumer to schedule another appointment in a proper timeframe. In another example, the medical records are updated and directly accessed to prompt the user.

Stage eight is essentially taking the service consumers back to the first stage in the dashboard in 150, just for a slightly different service. However, since the app already has input based on the summary the service provider gives, the app will be able to automate much of the previous stages. Going back to the diabetes example, the app already knows the service consumer needs to see a general physician every two months to review blood tests. Thus, the app now skips stages one through four and directly jumps the service consumer to confirming the appointment in stage five. The app will keep all these pieces of information organized and continue to send reminders to the service consumer so they can maintain pace with whatever they're supposed to be doing. With the app providing as much information and guidelines as possible, this opens up more time for the service provider to work on receiving other clients. Here is where the app really shows its efficiencies to both the service consumer and to the service provider for schedule and paying services.

### HOW TO MAKE THE INVENTION

### Computer Program Products and Methods

Embodiments of the present invention may be implemented in various ways, including as computer program products that comprise articles of manufacture. A computer program product may include a non-transitory computer-readable storage medium that is storing applications, programs, program modules, and/or the like. This non-transitory computer-readable storage media include all computer-readable media including volatile and non-volatile media.

In one embodiment, a non-volatile computer-readable storage medium may include a hard disk and/or the like.

In one embodiment, a volatile computer-readable storage medium may include random access memory (RAM), dynamic random access memory (DRAM), static random access memory (SRAM), cache memory (including various levels), flash memory, and/or the like. It will be appreciated that other types of computer-readable storage media may be substituted for or used in addition to the computer-readable storage media described above.

Various embodiments of the present invention may be implemented as methods, apparatus, systems, computing devices, computing entities, and/or the like. Thus, embodiments of the present invention may take the form of an entirely hardware embodiment, an entirely computer program product embodiment, and/or an embodiment that comprises combination.

Embodiments of the present invention are described below with reference to block diagrams. It should be understood that the block diagrams support various combinations of embodiments for performing the specified instructions, operations, or steps.

### System Architecture

FIG. 3 provides an illustration to one embodiment of the present invention. As shown in FIG. 3, this particular embodiment may include one or more management computing entities 300, one or more networks 320, and one or more user computing entities 310. Each of these components may be in direct or indirect communication with one another over the same or different wired or wireless networks.

As should be appreciated, the management computing entities 300 can include connections to service provider management computing entities. Furthermore, user computing entities 310 can include both service consumers and service providers in addition to those supporting the app.

Additionally, it should be appreciated that the various embodiments are not limited to this particular architecture.

### Management Computing Entity

FIG. 4 provides a diagram of a management computing entity 300 according to one embodiment of the present invention. In general, the terms computing entity, computer, entity, device, system, and/or the like. For example, one or more computers, computing entities, desktops, mobile phones, tablets, notebooks, laptops, watches, glasses, ear pieces, and servers or server networks. These perform the functions, operations, and/or processes may include transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

In one embodiment, the management computing entity 300 may also include one or more communications interfaces 430 for communicating with various computing entities, such as by communicating data, content, information, and/or similar terms used herein interchangeably that can be transmitted, received, operated on, processed, displayed, stored, and/or the like.

As shown in FIG. 4, in one embodiment, the management computing entity 300 may include or be in communication with one or more processing elements 410 (also referred to as processors, processing circuitry, and/or similar terms used herein interchangeably) that communicate with other elements within the management computing entity 300. It should be appreciated, the processing element 410 may be incorporated in a number of different ways and may be configured for a particular use or configured to execute instructions stored in volatile or nonvolatile media or otherwise accessible to the processing element 410.

In one embodiment, the management computing entity 300 may be in communication with non-volatile media. In one embodiment, the non-volatile storage or memory may include one or more non-volatile storage or memory media 420, including but not limited to hard disks, SD memory cards, and/or the like. As will be appreciated, the non-volatile storage or memory media may store databases, data, applications, modules, and/or the like.

In one embodiment, the management computing entity 300 may be in communication with volatile media and may also include one or more volatile memory media 440, including but not limited to RAM, DRAM, SRAM, cache memory, register memory, and/or the like. As will be appreciated, the volatile memory media may be used to store at least portions of the databases, data, applications, modules, and/or the like being executed by the processing element 410 in order to control certain aspects of the operation of the management computing entity 300 with the assistance of the processing element 410 and operating system.

In one embodiment, the management computing entity 300 may also include one or more communications interfaces 430 for communicating with various computing entities by communicating data, content, information, and/or the like (including transmitting, receiving, operating on, processing, displaying, storing, and/or the like). Such communication may be executed using a wired data transmission protocol or via wireless external communication networks using any of a variety of protocols.

It should be appreciated that the management computing entity 300 may be in communication with one or more input elements, such as a keyboard input, a mouse input, a touch screen/display input, motion input, movement input, audio input, and/or the like. The management computing entity 300 may also include or be in communication with one or more output elements (not shown), such as audio output, video output, screen/display output, motion output, movement output, and/or the like.

It should be appreciated that one or more of the management computing entity' s 300 components may be located remotely from other management computing entity 300 components and may include service provider entities. It should also be appreciated that these architectures and descriptions are provided for illustrative purposes only and are not limiting to the various embodiments.

### User Computing Entity

A user may be a service consumer, a service provider, someone supporting the app and/or the like. A user may operate a user computing entity 310 that includes one or more components that are functionally similar to those of the management computing entity 300. FIG. 3 provides a diagram of a user computing entity 310 that can be used with embodiments of the present invention. In general, a computing entity may refer to one or more computers, computing entities, desktops, mobile phones, tablets, phablets, notebooks, laptops, watches, glasses, servers or server networks, and/or any combination of devices in order to perform the functions, operations, and/or processes described herein. User computing entities 310 can be operated by various parties including a service consumer, a service provider, and/or someone supporting the app. The user computing entity 310 can include an antenna 540, a transmitter 510, a receiver 520, and a processing element 530 that provides signals to and receives signals from the transmitter 510 and receiver 520, respectively.

Since the signals provided to and received from the transmitter 510 and the receiver 520 may include signaling information in accordance with air interface standards of applicable wireless systems, the user computing entity 310 may be capable of operating with one or more air interface standards, communication protocols, modulation types, and access types. It may be appreciated that the user computing entity 310 may operate in accordance with any of a number of wireless communication standards and protocols, such as those described above with regard to the management computing entity 300. In a particular embodiment, the user computing entity 310 may operate in accordance with multiple wireless communication standards and protocols. In another embodiment, the user computing entity 310 may operate in accordance with multiple wired communication standards and protocols, such as those described above with regard to the management computing entity 300 via a network interface 570. It can be appreciated that these communication standards and protocols enable the user computing entity 310 to communicate with various other entities and that it can also download changes, add-ons, and updates, for instance, to its firmware, software (e.g., including executable instructions, applications, program modules), and operating system.

In one embodiment, the user computing entity 310 may include location determining modules, functionalities, and/or similar words used herein interchangeably. For example, the user computing entity 310 may include indoor and/or outdoor positioning aspects, such as a location module adapted to acquire, for example, latitude, longitude, altitude, geocode, course, direction, heading, speed, universal time (UTC), date, and/or various other information/data. Some of various position or location technologies may also include RFID tags, Wi-Fi access points, cellular towers, nearby computing devices and/or the like.

In one embodiment, the user computing entity 310 may also comprise an app (that can include a display 550 coupled to a processing element 530) and/or a user input interface (coupled to a processing element 530). For example, the app may be a user application, browser, user interface, and/or similar words used herein interchangeably executing on and/or accessible via the user computing entity 310 to interact with and/or cause display of information from the management computing entity 300, as described herein. The app can comprise any of a number of devices or interfaces allowing the user computing entity 310 to receive data, such as a keypad 560 (hard or soft), a touch display, voice/speech or motion interfaces, or other input device. In embodiments including a keypad 560, the keypad 560 can include (or cause display of) the conventional numeric (0-9) and related keys (#, *), and other keys used for operating the user computing entity 310 and may include a full set of alphabetic keys or set of keys that may be activated to provide a full set of alphanumeric keys. In addition to providing input, the app can be used, for example, to activate or deactivate certain functions, such as screen savers and/or sleep modes.

The user computing entity 310 can also include volatile storage or memory 580 and/or non-volatile storage or memory 590, which can be embedded and/or may be removable. For example, the non-volatile memory may be ROM, SD memory cards, and/or the like. The volatile memory may be RAM, DRAM, cache memory, and/or the like. The volatile and non-volatile storage or memory can store databases, data, applications, programs, program modules, and/or the like to implement the functions of the user computing entity 310. As can be appreciated, this may include a user application that is resident on the entity or accessible through a browser or other user interface for communicating with the management computing entity 300 and/or various other computing entities.

In another embodiment, the user computing entity 310 may include one or more components or functionality that are the same or similar to those of the management computing entity 300, as described in greater detail above. As will be recognized, these architectures and descriptions are provided for exemplary purposes only and are not limiting to the various embodiments.

### Definitions

As used herein, the term “service consumer” refer to anyone wishing to schedule, confirm, and pay for services from a service provider.

As used herein, the term “service provider” refers to real estate agents, real estate brokers, dentist, doctor, therapist, accountant, attorney, financial planner, mortgage brokers, mortgage lenders, insurance agent, title companies, title closers, home inspector, stager, photographer, home improvement contractor, and/or the like.

As used herein, the terms “data,” “content,” “digital content, “information,” and similar terms may be used interchangeably to refer to data capable of being transmitted, received, and/or stored in accordance with embodiments of the present invention. It will be appreciated that where a computing device is described herein to receive data from another computing device, the data may be received directly from another computing device or may be received indirectly via one or more intermediary computing devices/entities, sometimes referred to herein as a “network.” It will also be appreciated that where a computing device is described to transmit data to another computing device, the data may be sent directly to another computing device or may be sent indirectly via one or more intermediary computing devices/entities, such as, for example, one or more servers, and/or the like.

As used here in, the term “likelihood” refers to a measure of probability for occurrence of a particular event.

The term “machine learning model” refers to a machine learning task. Machine learning is a method used to devise complex models and algorithms that lend themselves to prediction. A machine learning model is a computer-implemented algorithm that can learn from data without relying on rules-based programming. These models enable reliable, repeatable decisions and results and uncovering of hidden insights through machine-based learning from historical relationships and trends in the data. In one embodiment, the machine learning model is a regression model.

In one embodiment, data received from a user computing entity is applied to a trained machine learning model to predict a services required.

The term “target service” refers to a value that a machine learning model is designed to predict. In the present embodiments, historical data is used to train a machine learning model to predict the target service. Historical observations of the target services are used for such training.

As used here in, the terms “dataset” and “data set” refer to a collection of data. A data set can correspond to the contents of a single database table, or a single statistical data matrix, where every column of the table represents a particular variable, and each row corresponds to a given member of the data set in question.

As used here in, the terms “database” refer to database, database instance, database management system, and/or similar terms used herein interchangeably may refer to a collection of records or data that is stored in a computer-readable storage medium using one or more database models.

As used here in, the term “non-volatile media” refers to non-volatile storage, memory, memory storage, memory circuitry and/or similar terms used herein interchangeably.

As used here in, the term “volatile media” refers to volatile storage, memory, memory storage, memory circuitry and/or similar terms used herein interchangeably.

As used here in, the term “computing entity” refers to device, system, computing entity, entity, and/or similar words used interchangeably may refer to one or more computers, computing entities, desktops, mobile phones, tablets, phablets, notebooks, laptops, watches, glasses, ear pieces, wristbands and/or any combination of devices or entities adapted to perform the functions, operations, and/or processes described herein.

### HOW TO USE THE INVENTION

### System Operation

FIG. 2 provides an illustration for a typical user and service provider interaction.

Selecting a specific service is the second stage in FIG. 2 item 200. This stage is used to refine and filter what the user is looking for. After the service consumer inputs a key word in the search function, the app will generate results based off this input. The service consumer can filter on categories like location, rating quality, and availability in 205. This stage is necessary to provide the service consumer with efficiencies for receiving the service. By filtering on service providers with immediate openings, the service consumer can quickly fill their need. By filtering on rating, the service consumer can be assured they are receiving the top service. The app will utilize the service consumer and the service provider's locations to aid in filtering on location should the service consumer want to filter on it. This involves the use of GPS. Rating quality is based off an algorithm found within the app that takes prior service consumer's ratings into account (this process is elaborated more in stage seven, where the service consumers and service providers rate one another). In embodiments the pairing of the service consumer and the service provider is based on their profile as well as their prior service ratings. In embodiments the pairing is based on service provider ratings for service consumers similar to the service consumer. In embodiments, the rating are based on strategic and tactical drivers and their likelihood to lead to a positive Net Promoter Score Rating. In embodiments, the pairing is based on GPS proximity.

The third stage, checking the calendar is a crucial step in connecting the service consumer with the service provider in 210. It is here where the app uses its own calendar feature that takes input from the service consumer on what specific time/date they're looking to use a service. This time preference from the service consumer filters to the calendar on the service provider's end to match up time periods/dates. The availabilities will appear to the service consumer where they can then confirm the time that matches with their preferences and the service provider's openings in schedule.

If the service provider doesn't have an availability within the time period that the service consumer desires, they will be prompted with options to either select a new time period or to seek a different service provider. For selecting a new time period, the app will tell the service consumer when the service provider has their next availability. In the case of seeking a new service provider, the app will provide suggestions on different service providers based on the criteria that the service consumer input in step number two (such as rating or location).

Also within stage three, the app will have a feature where the service provider can block off time when they're not open or don't want to receive any customers from the service consumer base. This is especially necessary so a service provider's whole calendar isn't booked full with service consumers trying to book their service requests. There will be an option to select windows of time that is completely customizable to the service provider. As an example, (the possibilities are completely up to the service provider) one service provider might want to have their window be open for 12 hours Monday-Thursday. Another might choose to work for 4 hours a day, only on the weekend. Those that are open for more hours will often time get more service consumers just based on sheer numbers/opportunities to match with a service consumer's time preference. However, some who are the highest rated might find themselves in most demand so they could adjust their hours/pricing accordingly.

If scheduling with multiple service providers, the service consumer will see the optimum time periods. If there is a sequence for the appointments, the service consumer will see in what order the appointments must be.

Stage number four in 215, providing a description of the issue, is a simple way to create efficiencies on both the service consumer's end and the service provider's end. A quick two sentence summary describing the problem and what the service consumer seeks goes a long way in aiding the service provider to help out in the best way possible. This way the service provider can start to plan out a course of action to help the service consumer long before the service consumer even meets the service provider face to face. This creates less time waiting for the service consumer and can create more inflow of service consumers for the service provider.

As an example, the founder of a start-up company wants to meet with a lawyer about incorporating their business. They have no clue what the process is and how much money it could be potentially be. They have already selected one of the top rated lawyers and connected on a time based off the first three stages (mentioned above). Meeting with the lawyer is going to be extremely beneficial even just meeting as is. However, if the service consumer provides the lawyer with a few details such as what type of start up it is, where they want to incorporate, whether they want to be a LLC or a C-corp, etc. this can tremendously improve the productivity and the meeting and cut the meeting time down. The meeting time being shortened saves value for the service consumer as most of these lawyers are paid hourly (along with many more professions). The service provider also has the potential to capture more revenue as they can work with more clients in a given amount of time and gain more money through numbers as opposed to individual sessions.

Stage five in 225, officially scheduling and confirming appointment, is simply what it sounds like. Here, the service consumer has already selected the service, selected an available time, and described the service needed in a little more depth. The first initial tracking screen has already told them which stage in the process the service consumer is in. After the service consumer confirms the time (that is also available on the service provider's calendar) then this meeting will be set for whatever time period was selected. This could be the same day or months in advance. The meeting will then appear on the service provider's calendar. In embodiments, the service consumers is provide expert knowledge of best practices to they are better informed for the service experience.

If scheduling and confirming multiple appointments, the service consumer selects teaming the recommended schedules and simultaneously confirms all appointments with a single calendar “check-out”.

To caveat the notion that service provider's will be caught off guard with last minute requests, the app fulfills these gaps. If a service provider has made themselves available for a block of time, but no one has booked yet then the service provider would be waiting for that spot to be filled right anyway. They'll be happy that someone has scheduled to meet with them during a time period when no one had yet done so.

The app will also provide updates to both the service provider and the service consumer. These updates can come in the form of cancellations or changes, interim steps that may be necessary, and reminders of an upcoming meeting. For example, service providers cannot cancel appointments without losing premium status for a period of time, for example 12 months. Service providers set period of refund and penalty fee within their profile and this is communicated to the service consumer prior to scheduling the appointments. A service provider will set how many days/hours they allow cancellations and changes without this affecting the service consumer. The same applies to the service consumer. These safeguards are just to make sure no one is deploying fraud or scams to block off competitor's schedules and then simply cancelling appointments. There are some small ramifications if a cancellation or change does occur within 24 hours of a meeting (regardless of the settings and this 24 hour period is for example only). Interim steps are items an service consumer can perform prior to meeting with the service provider without their aid.

Stage six in 235, paying for the service, is obviously one of the most important of the eight. It's the reason why service providers do what they do and why customers must seek out service providers in the first place. However, this stage can be a little nuanced based off which industry the transaction took place in (i.e. real estate as opposed to medical). Some industries the norm is that the service provider gets paid following the completion of some event (in the case of real estate the buying or selling of a home). However, in some industries the service provider is paid even before meeting with the client. The app will understand these different trends in each industry and make sure the norm is followed. In cases where insurance becomes a factor, the app will collect the service consumer's insurance information prior to the meeting so payment can be collected (which would be an interim step in stage five). In embodiments, payment can include mobile wallet technologies.

The app tracks the purchase of services across multiple service provider web sites and/or calendar software modules. The embodiment comprises a calendar cart software module which records services and dates selected by the service consumer from the multiple web sites and/or calendar software modules within a personal calendar cart. As discussed above, the present invention provides a system for maintaining a unified calendar cart that stores service information associated with service referrals from multiple service provider web sites and/or calendar software modules, and keeps track of the sources of such referrals. One benefit of this feature is that it enables the service consumer to perform a single calendar “check-out” to schedule and purchase services from multiple service provider web sites and/or calendar software modules. Additionally, this feature allows the app to accurately track and credit each service provider, on a per-appointment basis. For example, if, upon “check-out” from the app, the service consumer has three booked appointments in the calendar cart, each of which resulted from a referral from a different service provider website and/or calendar software modules, each service provider will be credited for its respective transaction fee.

Service providers will be able to customize payment somewhat. Say a real estate agent feels they're extremely well-versed in the current marketplace and they want to charge money just for showing buyers homes (in addition to the commission at the end). They can choose a price simply for taking service consumers on showings. This might seem like a flawed feature, but these service providers will know that not as many service consumers will be willing to pay that way and so they're list of clientele will go down. If their list goes down, they must achieve a spectacular rating so that people will be willing to pay them per showing knowing that they do a good job of connecting buyers with the homes they want to buy. In addition, service consumers can simply filter out these agents who pay per showing to cater it to more of the traditional style.

The seventh stage, the service consumer and the service provider rate one another, in 255 occurs after the service has been performed. In embodiments, the app will use a 10 star system, with 10 being the most outstanding based on the services strategic and tactical drivers. Each of the tactical drivers is a question after the completion of the service delivery with the 10 star system with 1 star being and very unsatisfied and 10 being very satisfied. In addition to these questions is the core Net Promoter Score (NPS) is an index ranging from −100 to 100 that measures loyalty of a first party (e.g., service consumer) to a second party (e.g., service provider). The NPS is based on a single question: “on a scale of 0-10, how likely is it that you would recommend [company name] to your friends, family or business associates.” Respondents who indicate a 6 or below are “detractors,” those who indicate a score of 7-8 are “passives,” and those who indicate a score of 9-10 are “promoters.” Calculating the NPS from the survey responses involves a procedure of subtracting the percentage of detractors from the percentage of promoters. These scores are then used as a way to filter on the quality of service or customer that the service consumer and service provider are looking for, respectively. There will be a feature that shows how many reviews either one has received throughout their time using the app. This will allow others to determine if a score was simply a one-off or if that's how the average has been for multiple reviews. This allows for further filtering for both the service consumer and the service provider.

This review process also has a private and a public component. A public review will be displayed for everyone to see. A private one is sent only to the service consumer or service provider. Often times, these will be ways to improve or tips that they can use. Distinguishing these can allow for a more fair assessment that doesn't overly deflate or inflate a particular review.

In embodiments, the eighth and final stage is predicting and tracking the next steps for the service consumer in 265 (and service provider, if necessary). When the meeting is concluded, the app prompts the service provider to briefly summarize the meeting so the app can determine the next steps necessary. This is an extremely useful stage in the process and gives the app a lot of functionality throughout industries. Being able to to provide the service consumer with prompts and suggestions for the next stage is invaluable in saving them time and being a one-stop app for services.

As an example, in the medical industry, if a doctor diagnoses the service consumer with diabetes, then the doctor will put this in the summary at the end of the meeting (along with specifics like the type, severity, etc.). The app will then prompt the service consumer with notifications about dietary restrictions, where to go to find the proper medicine, and other items such as exercise programs and nutritional health books. If the service consumer needs to see continued treatment from a doctor/specialist the app will prompt the service consumer to schedule another appointment in a proper timeframe.

Stage eight is essentially taking the service consumers back to the first stage in 150 which updates the dashboard in 150, just for a slightly different service. However, since the app already has input based on the summary the service provider gives, the app will be able to automate much of the previous stages. Going back to the diabetes example, the app already knows the service consumer needs to see a general physician every two months to review blood tests. Thus, the app now skips stages one through four and directly jumps the service consumer to confirming the appointment in stage five. The app will keep all these pieces of information organized and continue to send reminders to the service consumer so they can maintain pace with whatever they're supposed to be doing. With the app providing as much information and guidelines as possible, this opens up more time for the service provider to work on receiving other clients. Here is where the app really shows its efficiencies to both the service consumer and to the service providers for scheduling, confirming, and paying for services.

### CONCLUSION

Many modifications and other embodiments of the disclosures set forth herein will come to mind to one skilled in the art to which these disclosures pertain having the benefit of the teachings presented in the foregoing description and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation, unless described otherwise.

1. A computer-implemented method for pairing service consumers and service providers, comprising:
receiving, using a processor, a plurality of data over a network from service consumer and service provider computing entities;
receiving, using a processor, a plurality of service consumer and service provider data from a database;
programmatically associate, using a first trained machine learning model, each service consumer with service providers; and
for each service consumer, programmatically generate service provider pairing based in part on service consumer data input from their computing entity.

2. The computer-implemented method of claim 1, wherein a programmatically generated pairing of a service consumer with a service provider is based in part on location and availability maintained in a database.

3. The computer-implemented method of claim 1, wherein a programmatically generated pairing of a service consumer with a service provider is based on their profiles maintained in a database.

4. The computer-implemented method of claim 1, wherein a programmatically generated pairing of a service consumer with a service provider is based on their ratings maintained in a database.

5. The computer-implemented method of claim 1, wherein a programmatically generated pairing of a service consumer with a service provider is based on service consumers with similar profiles maintained in a database.

6. The computer-implemented method of claim 1, wherein a programmatically generated pairing of a service consumer with a service provider is based on their ratings which are based on strategic and tactical drivers and maintained in a database; and
programmatically generate their likelihood to lead to a positive Net Promoter Score Rating using a second trained machine learning model.

7. The computer-implemented method of claim 1, wherein a programmatically generated pairing of a service consumer with a service provider is based on service consumers with similar profiles the pairing is based on GPS proximity received over a network from their computing entities.

8. A computer-implemented method for service consumers to use their computing entity to enter data and programmatically paired with service providers based on their availability maintained in a database; and
programmatically generate a confirmation with the service provider using a network to communicate their computing entity.

9. The computer-implemented method of claim 8, wherein a service consumer will be notified on their computing entity when a service provider is not available; and
programmatically generate alternate times the service provider is available; and
programmatically generate alternate service provider pairings.

10. The computer-implemented method of claim 8, wherein a service provider can enter data into a database to block off times and dates for availability.

11. The computer-implemented method of claim 8, wherein a service consumer can schedule multiple service providers simultaneously based on their individual availabilities maintained in a database.

12. The computer-implemented method of claim 8, wherein a service consumer can schedule multiple service providers simultaneously in a required sequence based on their individual availabilities maintained in a database and a third trained machine learning model.

13. A computer-implemented method for service consumers to use their computing entity to enter data regarding services required and programmatically generate a notification with the service provider using a network to communicate to their computing entity.

14. A computer-implemented method for a service consumer and a service provider to use their computing entities to confirm an appointment maintained in a database.

15. The computer-implemented method of claim 14, programmatically generated recommendations for the service consumer and service provider using a fourth trained machine learning model;
and communicated to the service consumer and service provider computing entities; and maintained in a database.

16. The computer-implemented method of claim 14, for a service consumer to simultaneously confirm multiple appointments with multiple service providers; and
programmatically generating multiple requests to service providers using a single request from the service consumer computing entity.

17. The computer-implemented method of claim 14, wherein data is communicated to the service consumer and service providers computing entities based in part on new availability data entered into a database by the service consumer and service providers using their computing entities.

18. The computer-implemented method of claim 14, wherein data is communicated to the service consumer and service providers computing entities based on programmatically generated reminders.

19. The computer-implemented method of claim 14, wherein data is communicated to the service consumer and service providers computing entities based programmatically generated tasks to complete before the schedule appointment.

20. The computer-implemented method of claim 14, wherein the service consumer is billed for cancelling an appointment with a service provider based on a profile maintained in a database.

21. A computer-implemented method for a service consumer to be programmatically billed electronically for a service provided.

22. The computer-implemented method of claim 21, wherein the service consumer is billed prior to the service provided based on a profile maintained in a database.

23. The computer-implemented method of claim 21, wherein the service consumer health insurance information is collected prior to the appointment using a fifth trained machine learning model.

24. A computer-implemented method for a service providers and consumers to be programmatically track calendars for scheduled appointments and availability.

25. The computer-implemented method of claim 24, wherein the service consumer is programmatically able schedule multiple appointments with multiple service providers simultaneously in a single “check-out”.

26. The computer-implemented method of claim 24, wherein the service consumer “check-out” programmatically generates referral fees for the service providers.

27. The computer-implemented method of claim 24, wherein the service consumer “check-out” programmatically generates service fees for the service consumers.

28. A computer-implemented method for a service providers and consumers to be programmatically rate one another.

29. The computer-implemented method of claim 28, wherein the service provider and service consumer rating programmatically generating their respective NPS rating using a ten point scale; and
programmatically generating strategic and tactical driver questions.

30. A computer-implemented method for service providers to be programmatically notified to enter a summary of the meeting.

31. A computer-implemented method for service providers and consumers to be programmatically notified of the next steps to be completed.

32. A non-transitory computer-readable storage medium, the computer-readable storage medium including instructions that when executed by a computer, cause the computer to:
pair service consumers and service providers, comprising:
receiving, using a processor, a plurality of data over a network from service consumer and service provider computing entities;
receiving, using a processor, a plurality of service consumer and service provider data from a database;
programmatically associate, using a first trained machine learning model, each service consumer with service providers; and
for each service consumer, programmatically generate service provider pairing based in part on service consumer data input from their computing entity.
